(19) United States
(12) Patent Application Publication
(10) Pub. No.: US 2008/0112998 A1
(43) Pub. Date: May 15, 2008

(54) Innovative bottom-up cell assembly approach to three-dimensional tissue formation using nano-or micro-fibers

(76) Inventor: Hongjun Wang, Nutley, NJ (US)

Correspondence Address: Hongjun Wang;Apt C, 29 Lovel Court, Nutley, NJ, 07110, US

(21) Appl. No.: 11/985,273

(22) Filed: Nov. 14, 2007

Related U.S. Application Data
(60) Provisional application No. 60/865,743, filed on Nov. 14, 2006.

(51) Int. Cl. A61F 2/02; A61K 35/12; A61P 43/00

(52) U.S. Cl. 424/423; 424/93.7

(57) ABSTRACT

The present invention provides a synthetic tissue scaffold, the scaffold comprising alternating layers of electrospun polymers and mammalian cells sandwiched within. A novel method is also provided for generating a three-dimensional tissue by electrospinning polymers and seeding cells in alternating layers on an aqueous solution in a desired shape. This invention is suitable for generating animal tissue as well as for delivery of drugs or other substances to a recipient.

### FIELD OF THE INVENTION

The present invention relates to bioengineered three-dimensional tissue formation using microsize or nanosize fibers and to methods of producing such microparticles. The formed three-dimensional tissues are useful in replacement or repair of damaged mammalian tissues and organs.

### BACKGROUND OF THE INVENTION

Tissue engineering and its challenges. Tissue and organ trauma, failure or dysfunction due to congenital deformities, accident, cancer, or aging often requires surgical treatment to restore function, since most tissues cannot regenerate when injured or diseased. Even for tissues that are able to regenerate spontaneously (e.g. angiogenesis (vascular tissue), osteogenesis (bone) and chronic wound healing), there exists a critical size of defect for repair, above which tissue regeneration and healing are inhibited. A critical size for bone defects of 6×10 mm and 15×25 mm was found, for example, in in vivo studies on pig sinus (Brodkin K R, et al. Biomaterials 25: 5929-5938, 2004) and rabbit femoral condyles (Brodsky V Y, Biol Rev Camb Philos Soc 81: 143-162, 2006), respectively.

One conventional way in which to recover the function of diseased tissue is to use prosthetic devices or transplants (Cai K, et al. Biomaterials 26: 5960-5971, 2005) (the “prosthetic approach”). However, prosthetic devices do not permanently replace the full and proper function of the damaged tissue or organ and cannot prevent continued progression of disease (Christopher R A, et al. Int J Mol Med 5: 575-581, 2000). Autograft has been considered as a “gold standard” treatment (Croll T I, et al. Biomacromolecules 7: 1610-1622, 2006) (the “transplantation approach”). However, lack of donor sites and aroused morbidity has greatly restricted its application. Therefore, there is a compelling need for other alternatives to restore, repair, and/or maintain tissues and organs.

The emerging field of tissue engineering has proven to be a very promising alternative to both the prosthetics and transplantation approach (Albrecht D R, et al. Nat Methods 3: 369-375, 2006). The tissue engineering approach typically involves seeding cells in three-dimensional scaffolds and in vitro or in vivo culturing of cell-enriched scaffolds to form tissues. This approach has demonstrated clinical success, as evidenced by commercially available tissue engineered skins (see, e.g., Cutler S M, et al. Biomaterials 24: 1759-1770, 2003, De Rosa M, et al. J Cell Physiol 198: 133-143, 2004). However, several challenges remain: first, the majority of currently available tissue engineered grafts are composed of one cell type and have simple morphological structure. This is different from natural tissues with multiple cell types and complicated matrix structure and composition (De Santis G, et al. Plast Reconstr Surg 113: 88-98; discussion 99-100, 2004). Second, limited cell migration and limited tissue ingrowth capacity also restrict the maximal size of tissues that can be created by this approach. For the latter challenge, improved cell initial distribution in the scaffold with high seeding density can increase the tissue ingrowth to a certain extent (El Ghalbzouri A, et al. Wound Repair Regen 12: 359-367, 2004). However, the preferential tissue growth in the periphery of scaffold as a result of gradient nutrient distribution is persistently problematic. The best solution to this peripheral tissue growth pattern is vascularization of the cultured tissue to eliminate the interior ischemia or hypoxia (Fang N, et al. Macromol Biosci 5: 1022-1031, 2005). Additionally, the preformed vasculature can accelerate host integration after implantation, which is an important issue for the survival of implants and tissue function restoration as well.

Critical role of 3D cell organization in tissue formation. In most tissues, cells are embedded in the entangled extracellular matrix network (ECM). Although the cellular component constitutes only a small portion of the whole tissue, it is the most critical contributor to synthesizing and defining the tissue composition and properties. Proper cell phenotype is of particular importance in regulating matrix biosynthesis and remodeling (Hamdan M, et al. Clin Implant Dent Relat Res 8: 32-38, 2006). It is evident that spatial arrangement of cells embedded in ECM has a great effect on the phenotypic fate of these cells. For example, isolated chondrocytes lose their round morphology in monolayer culture and turn into a fibroblast-like phenotype (Han X, et al. Oncogene 20: 7976-7986, 2001). This phenotypic change leads to a biosynthetic alteration of matrix proteins, increasing the synthesis of collagen type I rather than collagen type II, a chondrogenic marker protein (Harimoto M, et al. J Biomed Mater Res 62: 464-470, 2002). However, upon three-dimensional culture in a cell pellet or in an agarose hydrogel, the fibroblastic chondrocytes redifferentiate into a chondrogenic phenotype and show elevated synthesis of both collagen type II and glycosaminoglycans (Harimoto M, et al. J Biomed Mater Res 62: 464-470, 2002, Ito A, et al. Tissue Eng 10: 873-880, 2004).

During the phenotypic transformation of ECM-embedded cells as a result of the spatial arrangement of the cells, the interactions between cells and cell-matrix both play an important role by regulating different gene expression. Unlike epithelial cell types that prefer a two-dimensional surface for growth, most other types of cells favor a three-dimensional microenvironment. For these non-epithelial cell types, the microscale contact and communication between cells and cues from the matrix cause sequential intracellular events to influence cellular behavior (Ito A, et al. Tissue Eng 11: 489-496, 2005).

The adhesion of cells onto the matrix is regulated by adhesion receptors on the cell membrane. It has been reported that the cell-to-collagen fibril affinity is essential for the phenotypic performance of bioartificial myocardial tissue equivalents (Jakob M, et al. J Cell Biochem 81: 368-377, 2001). Collagen fibrils provide the major biomechanical scaffold for cell attachment and anchorage of macromolecules, allowing the shape and form of tissues to be defined and maintained. Among adhesion receptors, integrins have proven to be essential mediators, participating in a wide-range of biological interactions including tissue development by regulating cell-cell and cell-matrix contacts (Janekovic A, et al. J Colloid Interface Sci 103: 436-447, 1985).

Intercellular communication also significantly affects tissue formation by influencing cellular behavior via direct contact (e.g., gap junction), and the exchange of bioactive molecules (e.g., growth factors). Intercellular communication is greatly influenced by intercellular distance. In a recent report, it was shown that the biosynthesis of chondrogenic marker proteins was elevated when cultured in hydrogel with inter-chondrocyte distance less than 10 μm but was inhibited in the chondrocyte pellet culture with direct cell contact (Katsuko S. et al. Materials Science and Engineering: C 24: 437-440 2004). This observation indicates that certain intercellular distance is necessary for non-connected cell types to maintain the proper cellular activity. Additionally, tissues or organs normally are composed of multiple cell types. Each type of differentiated cell has its own well-defined spatial organization to achieve specific function, and also affects neighboring cells by cell-cell communication or through secreted soluble factors (Kharlampieva E., et al. Macromolecules 36: 9950-9956, 2003). The coexistence of multiple cell types mutually regulates the cellular activity, e.g., epidermal homeostasis is controlled by mutual keratinocyte-fibroblast interaction apart from paracrine acting factors (Kharlampieva E., et al. Langmuir 20: 9677-9685, 2004). Therefore, three-dimensional cell arrangement allowing the presentation of interdependent cell types is necessary for proper tissue formation.

In tissue engineering, the spatial arrangement of cells on a micron scale plus the capability for heterogeneity of cell type and vascularization are desirable traits. However, currently designed scaffolds do not allow co-culture of multiple cell types with controllable intercellular separation.

“Cell sheet engineering” for tissue formation. To avoid the limitations in conventional tissue engineering such as restricted tissue ingrowth, stacks of cultured cell sheets recently have been explored to build tissues with or without the use of scaffold (Bet M R, et al. Biomacromolecules 2: 1074-1079, 2001) (Knosche C., et al. Particle and Particle Systems Characterization 14: 175-180 1997). FIG. 1 illustrates this approach. This strategy first was reported in 2002 in an attempt to fabricate cardiac muscle tissue (Bet M R, et al. Biomacromolecules 2: 1074-1079, 2001.) In this study, three-dimensional (3D) cardiac muscle tissue was obtained by directly stacking multiple layers of cultured myocyte sheets. Following this approach, other laminar structured tissues, such as liver lobules and kidney glomeruli have similarly been built (Kofidis T., et al. Med Eng Phys 26: 157-163, 2004) (Kunz-Schughart L A, et al. Am J Physiol Cell Physiol 290: C1385-1398, 2006). Because of the fragility of cultured cell sheets, porous polymeric thin membranes also were used to support cell growth in liver tissue reconstruction (Kofidis T., et al. Med Eng Phys 26: 157-163, 2004). The major advantages of this approach are: 1) integral tissue formation and 2) minimal use of scaffold. However, there are also a few drawbacks, such as: 1) limitation of the method to tissues with flat morphology, 2) requirement of an additional step to obtain cell sheets prior to stacking, 3) difficulties in thick tissue manufacturing due to the existence of interior ischemia or hypoxia, and 4) applicability to limited number of cell types.

Recent development of microfabrication technology enables the fabrication and modification of scaffolds on a micro- or nano-scale. Controlling the structure and composition of a scaffold in dimensions of several hundred microns is often adequate (Griffith, L. G. and M. A. Swartz Nat Rev Mol Cell Biol 2006. 7(3): p. 211-24) because cell-scaffold interaction is at a molecular level. Of the microscale technologies, electrospinning, a high voltage driven spinning technique, is particularly appealing due to its ability to produce nanofibers with similar dimensions as the collagen fibrils in tissue matrix (Pham, Q. P. et al. Tissue Eng 2006. 12(5): p. 1197-211).

Polymeric micro and nanofibers can be made using electrostatic spinning or electrospinning processes (U.S. Pat. No., 1,975,504, U.S. Pat. No., 2,160,962, U.S. Pat. No., 2,187,306 to Formhals, Baumgarten P K. J of Colloid and Interface Science 1971; 36:71-9; Reneker D H, Chun I. Nanotechnology 1996; 7:216-23). Briefly, electrospinning uses an electric field to draw a polymer solution from the tip of the capillary to a collector. A high voltage DC current is applied to the polymer solution which causes a jet of the polymer solution to be drawn towards the grounded collector screen. Once ejected out of the capillary orifice, the charged polymer solution jet gets evaporated to form fibers and the fibers get collected on the collector. The size and morphology of the fibers thus obtained depends on a variety of factors such as viscosity of the solution, polymer molecular weight, nature of the polymer and other parameters regarding the electrospinning apparatus. The electrospinning process to form polymer nanofibers has been demonstrated using a variety of polymers (Huang, et al. Composites Science and Technology 2003; 63).

Numerous polymers have been successfully electrospun into micro- and nanosized fibers using this technique. Dependent on solvents used, polymer concentration and spinning conditions, fiber diameter can range from several hundred nanometers to several micrometers. To mimic the native ECM, natural polymer is preferred.

The advantages of these filament scaffolds in controlling cell responses have been demonstrated in a number of studies (Petite, H., et al. Nat Biotechnol, 2000. 18(9): p. 959-63; Chua, K. N., et al. Biomaterials, 2005. 26(15): p. 2537-47; Li, C., et al., Biomaterials, 2006. 27(16): p. 3115-24; and Ji, Y., et al., Biomaterials, 2006. 27(20): p. 3782-92). However, some critical issues have constrained the wide application of electrospinning in tissue engineering, and among of them the most important one is that the inter-fiber spaces (that is, the pore size) in electrospun nano/micro-fibrous meshes are in the submicron meter range, which is difficult for cells to penetrate. Therefore, the cells often grow only on the surface of the meshes. In addition, the sterility of fibrous meshes is another concern in its application for cell growing substrate.

Accordingly, it is evident that a significant need exists for improved tissue regeneration techniques and materials since current treatments, while frequently effective, have a number of disadvantages.

### SUMMARY OF THE INVENTION

The present invention addresses the need for improved tissue regeneration techniques and materials.

The present invention provides a method to fabricate synthetic tissue scaffold on an aqueous surface, the scaffold comprising alternating layers of electrospun fibers, optionally incorporating at least one bioactive molecule, and cells.

The invention further provides a method for generating a three-dimensional tissue, the method comprising the steps:
a) Electrospinning a layer of electrospun polymers of the invention,
b) Depositing a layer of the electrospun polymers onto an aqueous surface in an arbitrary shape,
c) Depositing a layer of one or more living mammalian cells onto the layer of electrospun polymers,
d) Repeating steps b and c until achieving desired three-dimensional tissue size, where in step (c) the first type is optionally replaced with a different type of cell, to form the three-dimensional tissue.

### BRIEF DESCRIPTION OF THE DRAWINGS

FIG. 1 shows the currently applied “cell sheet engineering” strategy, as described in the review (Fang N., et al. Macromol Biosci 5: 1022-1031, 2005). Monolayer cultured cell sheets (A) are stacked to form 3D structure with monotypic cell layers (B) or heterotypic cell layers (C).

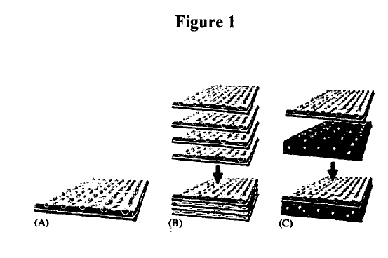

FIG. 2 shows cells embedded among collagen fibrils observed by transmission electron microscope (A) and a schematic diagram of layer-by-layer tissue generation (B). Fibers can be made of various materials, contains various drugs and having various geometrical parameters such as diameter or orientation. Cells can be single type or multiple types in different layers or the same layer as shown in different colors.

FIG. 3 shows a schematic illustration of in situ layer-by-layer tissue regeneration. In step 1, a layer of polymer fibers is deposited in the wire loop on the surface of medium by electrospinning (the thickness can be controlled by selecting the flow rate and collecting time). In step 2, certain cells are seeded on the surface of fibrous membrane. Steps 1 and 2 are repeated until the desired amount of layering is achieved. The inset B is a confocal image of the cross-section of multilayered tissue. Fibers are labeled as green using FITC-BSA, and cell nuclei stained blue using DAPI.

FIG. 4 shows that the layered tissue shape is determined by the shape of the metal wire loop, which can be any shape such as quadrangle, circular, triangle, and irregular, as indicated in (a). The cell seeding can also be extended to other available techniques such as cell spray. We have designed a new version for scaling up the process, which uses an automated head with multiple channels (b) for cell seeding, whereby the multi-channel head moves laterally. The final formed layered tissue can be further processed into different structures, e.g., rolled into a tube or a rod, or stacked into a thick sandwich or applied to any arbitrary surface (c).

FIG. 5 shows cell culture on different fibrous scaffolds (confocal). Fibroblasts cultured on polycaprolatone (PCL) and collagen-containing fibrous membrane were stained with pholloidin (red) for actin and DAPI (blue) for nuclei. Scale bar=20 μm.

FIG. 6 shows microscopy images indicating formed tissue using a layer-by-layer approach. (A) Light microscopy micrograph of methylene blue stained cross-sections of fiber-cell construct which was cultured for 24 hours after LbL tissue building. Dark blue stained cell nuclei. (B) Fluorescence micrograph of DAPI stained cross-sections of fiber-cell construct cultured for 24 hours. Nuclei stained as blue. (C) and (D) Confocal microscopy images of cross-sections of formed tissue. Fibers were labeled with FITC (stained green) and cells were stained blue. The thickness of the fiber layer can be easily adjusted, shown as (C) 10 μm and (D) 20 μm.

FIG. 7 shows the release of BSA from PCL fibrous meshes containing BSA. The BSA release was performed by incubating the mesh disc (1.3 cm in diameter) in 1 mL PBS solution and measuring the BSA amount in supernatant. BSA evenly distributed in PCL fibers as shown in the inset using FITC-labeled BSA under fluorescence microscope (Nikon).

FIG. 8 shows fluorescent images of the cross-section of sequentially deposited fibers with two different polymers (polymers A and B). The sequence was indicated as the diagram on the left, and fibers were collected on aluminum foil as the substrate.

FIG. 9 shows confocal microscopy micrographs of cell-fiber constructs cultured for 24 hours after seeding. A) Cross section view of three-layer structure with cells attached to the middle layer of fibers. * indicates material A fibers (FITC-labeled as green), which is less favorable for cell attachment compared to # material B fibers (no fluorescence). B) The top view of cells which attached to FITC-labeled material B fibers (green). C) Cross-section view of cell-fiber multilayered construct achieved by stacking 2 prepared multilayer structures of cells and FITC labeled material B fiber, which is morphologically close to muscle tissue. D) Cross-section view of a tubular structure from rolled cell/fiber (material B) sheet via sequentially layer-by-layer building. The cells used are human dermal fibroblasts and cell nuclei were stained blue using DAPI. Magnification of A and B is 20×, C and D is 10×.

FIG. 10 shows hematoxylin and eosin stained cross-section of cultured fiber-cell constructs. A) Formation of dermal tissue from fibroblast/electrospun fiber layered constructs for 7 days. Arrow head indicates dermal fibroblasts and asterisk shows fibers. Scale bar=50 μm. B) Formation of skin tissues composed of epidermal (E) and dermal (D) layer and formed by culturing “L-b-L” cell assembled constructs for 3 days. Green broken line outlines the border between E and D. Scale bar=50 μm.

### DETAILED DESCRIPTION OF THE INVENTION

Unlike conventional tissue engineering, where cells are seeded into and cultured in preformed porous scaffolds, the present invention uses microsize polymeric building blocks to provide cells with an adhesion platform and to form a three-dimensional tissue structure. These building blocks are capable of assembly with cells in aqueous solutions to form three-dimensional structure. Advantages and embodiments of this invention include: the capability of building tissues with arbitrary shape and size; the capability of assembling cells onto arbitrary interior or exterior surfaces; controlled growth and organization of multiple cell types; control of intercellular spacing by varying the size of polymeric building blocks; minimal need of scaffold materials; the capability of locally delivering soluble factors from polymeric building blocks to cells; and the capability of creating vasculature in cultured tissues.

“Micro-,” as used herein, i.e., such as in the term “microsize fiber,” generally refers to structures having dimensions that may be expressed in terms of micrometers. For example, the term “microscale structure” may refer to a structure having dimensions of about greater than 0 μm to about 999 μm, greater than 0 μm to about 500 μm, greater than 0 μm to about 100 μm, greater than 0 μm to about 50 μm, about 20 to about 50 μm, about 10 to about 20 μm, about 5 to about 10 μm, about 1 to about 5 μm, about 1 μm, or about 0.5 to about 1 μm. This definition is not meant to limit the invention; however, to such sizes, and as defined herein, “micro-” may also include “nano-” scale structures.

“Nano-,” as used herein, generally refers to structures having dimensions that may be expressed in terms of nanometers, or materials composed therefrom. For example, a nanoscale structure may refer to structures having dimensions of greater than 0 nm to about 999 nm, greater than 0 nm to about 500 nm, greater than 0 nm to about 100 nm, greater than 0 nm to about 50 nm, about 20 nm to about 50 nm, about 10 nm to about 20 nm, about 5 nm to about 10 nm, about 1 nm to about 5 nm, about 1 nm, or about 0.1 to about 1 nm.

### “Layer-by-Layer Cell Assembly” for Tissue Formation

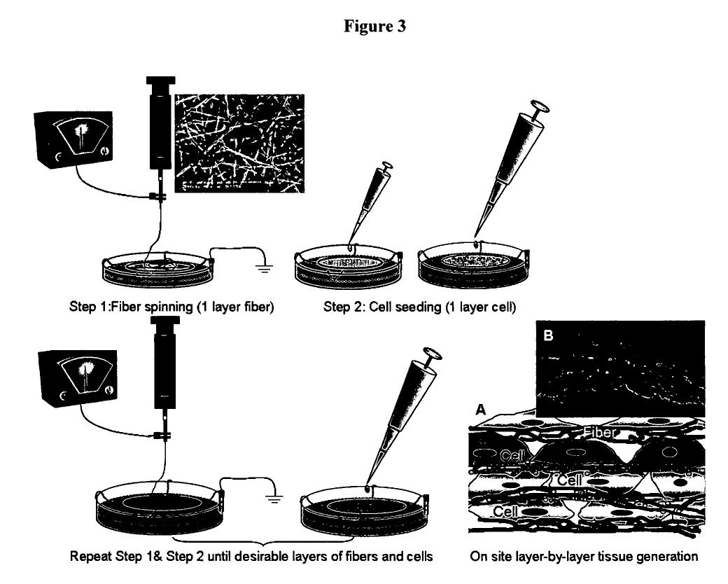

Most tissues or organs have a complex structure (typically stratified and lattice-like) and variable contour, especially with higher-order cell organization and heterogeneous cell types. To create or mimic this complex tissue structure, the present invention provides an innovative “bottom-up,” “layer-by-layer self assembly of cells” approach to reconstructing tissue (FIG. 3). “Bottom-up cell assembly,” as used herein refers to the construction of micro and nano structures and complex systems from atoms and molecules. This approach allows cell assembly into a three-dimensional structure with a highly ordered spatial cell arrangement. It allows separate co-culture of heterogeneous cells, microscale three-dimensional control of cell distribution and organization, and, most importantly, enables the vascularization of tissues. To facilitate the cell assembly, microsize and/or nanosize building blocks are fabricated on an aqueous surface. These building blocks include microsize or nanosize fibers.

Layer-by-Layer Tissue Generation Using Nanofibers or Microfibers:

The invention provides a synthetic tissue scaffold comprising alternating layers of electrospun microfiber or nanofiber polymers and cells. The cells may be any living cell, preferably of animal origin, and are most preferably living mammalian cells. The fibers optionally incorporate at least one bioactive molecule either residing within the fibers themselves or in between the fibers, i.e., within pores of the fibers.

The invention also provides a method for generating a three-dimensional tissue. This method comprises electrospinning and depositing a layer of microsize or nanosize electrospun polymers onto an aqueous surface in an arbitrary shape; depositing a layer of one or more living mammalian cells onto the layer of electrospun polymers. These steps are optionally repeated until the desired three-dimensional tissue size is achieved. The types of cells and fibers may be varied upon repeating the layers, so as to achieve a co-culture of multiple cell types with a specifically designed micro-environment, mimicking natural tissue.

“Tissue” is defined herein to refer to a group of cells with a specific function in the body of an organism. Examples of tissues found in some animals include, without limitation, lung tissue, vascular tissues, and muscle tissue. Tissues usually are composed of nearly identical cells and the intercellular substances surrounding them, and often are organized into larger units called organs.

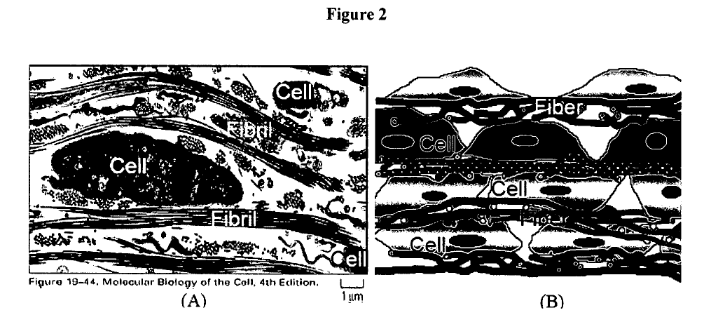

Growing thick 3D tissues is extremely complex. Such structures are hierarchical, heterogeneous and spatially organized arrangements of multiple cell types, extracellular matrices and more complex vascular and neural structures. A variety of tissues in our body are composed of layered structures, for instance, the epithelial layer on the inner surface of our internal organs and the endothelial lining in blood vessels. In addition, even for those tissues without clear layered structure such as connective tissues, cells are embedded in ECM fibrils (FIG. 2A), providing cells with the support and protection and conveying the external cues to cells.

Layer-by-layer tissue regeneration using nanosize and/or microsize fibers (as indicated in FIG. 2B) would not only allow morphological mimicking of the tissue structure, provide a 3D microenvironment similar to that in vivo, which is considered an advantage over unchanged universal environment in maintaining cell phenotype (Schindler, M., et al., Biomaterials, 2005. 26(28): p. 5624-31; Zong, X., et al. Biomaterials 2005. 26(26): p. 5330-8; Sun, T., et al. Tissue Eng, 2005. 11(7-8): p. 1023-33), but also would allow co-culture of multiple cell types and provide diverse scaffolds with different composition and morphology to specific type cells.

In this invention, on-site cell seeding is performed together with microsize or nanosize fiber electrospinning to achieve layer-by-layer tissue rebuilding, i.e., tissue regeneration (FIG. 2). This novel technique allows cells to penetrate into nano- and/or microfibrous meshes rather than grow only on, the surface of the meshes. In other techniques it is difficult for cells to penetrate the inter-fiber spaces (pore size) of theses electrospun meshes because the pore sizes are too small (in the micro or submicron meter range). The on-site tissue regeneration method takes place on the surface of an aqueous solution such as cell culture medium, where the mesh is hydrated continually during processing, and thereby preventing dehydration of the seeded cells on the fibrous mesh.

Direct electrospinning of fibers on, the surface of physiological solution coupled with on-site cell seeding to build tissue is a novel technique with the following advantages over conventional tissue engineering, i.e., seeding and culturing cells into a preformed porous scaffold: 1) flexibility in allowing heterogeneous cell types, 2) reduction of culture period for tissue formation as a result of even cell distribution and an appropriate micro-environment, 3) control of inter-cellular spacing by varying the thickness of the nano/microfibrous layer, 4) flexibility in providing a specific micro-environment, including the composition of fibrous scaffold, orientation, fiber diameters, mechanical properties, for targeted cells, 5) the potential implementation of local delivery of therapeutic agents or bioactive molecules such as growth factor or genes to cells, and 6) the potential to create vasculature in cultured tissues, which is critical in the clinical application of tissue engineered grafts.

“Therapeutic agent” refers to a substance, including a molecule, substance or compound of any type that, when administered to a subject in need thereof, alleviates one or more symptoms of a disease or undesired clinical condition, reduces the severity of a disease or clinical condition, prevents or lessens the likelihood of development of a disease or undesired clinical condition, or facilitates repair or regeneration of tissue in a manner other than simply providing general nutritional support to the subject. A therapeutic agent generally is administered in an effective amount, i.e., an amount sufficient to achieve a clinically significant result. A therapeutic agent may be a small molecule or a biomolecule, for example. See Goodman and Gilman's The Pharmacological Basis of Therapeutics, 10.sup.th Ed., and Katzung, Basic and Clinical Pharmacology, for examples.

As used herein the term “treating” includes abrogating, substantially inhibiting, slowing or reversing the progression of a condition, substantially ameliorating clinical or aesthetical symptoms of a condition, substantially preventing the appearance of clinical or aesthetical symptoms of a condition, and protecting from harmful or annoying stimuli.

“Subject” or “recipient” as used herein refers to an individual to whom an agent is to be delivered, e.g., for experimental, diagnostic, and/or therapeutic purposes. Preferred subjects are mammals, primates or humans.

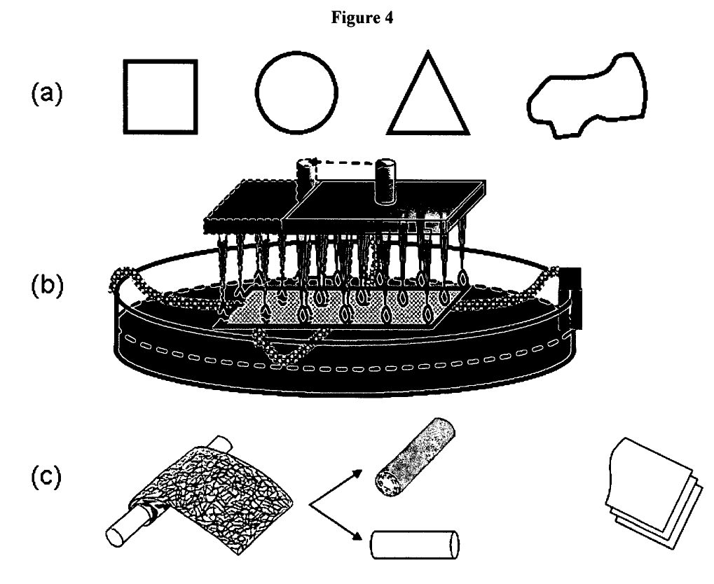

In this invention, the tissue shape created by the direct electrospinning of fibers on the surface of physiological solution together with on-site cell seeding is defined by a metal wire loop with a preferred outline shape. This loop can be any arbitrary shape (see FIG. 4a). We have used loops with square, rectangle, circular shape (see FIG. 4a) for electrospinning in our studies. The wire we used is platinum, but can be any conductive wire as long as it is noncytotoxic. In the assembled multilayered cell/fiber construct, the adjustable thickness between cell layers and highly interconnected pores of the fibrous layers will allow free cell-cell communication and nutrition transport, necessary to the regulation of cellular activity. Multiple polymers such as PLGA, PLLA, PCL, collagen, and gelatin have been successfully electrospun into fibers and tested for biocompatibility by culturing cells on surface. In addition, the cells were manually seeded in the preliminary studies; an automatic cell seeding unit with multichannels has been accordingly designed for upscale of the method as shown in FIG. 4b. The formed multilayered tissues can be further processed into different morphology such as rolled into rod or tube or stacked into thick tissue (FIG. 4c). The polymers used for this layer-by-layer tissue generation can be extended to any materials which are biocompatible and electrospinnable. Additives such as hydroxyapatite (HAp) can be included in the fibers as well.

“Electrospinning,” as used herein, refers to a process wherein a high voltage electric field is generated between oppositely charged polymer fluid contained in a glass syringe with a capillary tip and a metallic collection screen. As the voltage is increased, the charged polymer solution is attracted to the screen. Once the voltage reaches a critical value, the charge overcomes the surface tension of the suspended polymer cone formed on the capillary tip of the syringe and a jet of ultra-fine fibers is produced. As the charged fibers are sprayed, the solvent quickly evaporates and the fibers are accumulated randomly on the surface of the collection screen. This results in a nonwoven mesh of nano and micron scale fibers. Varying the charge density (applied voltage), polymer solution concentration, solvent used, and the duration of electrospinning can control the fiber diameter and mesh thickness. Other electrospinning parameters which may be varied routinely to affect the fiber matrix properties include distance between the needle and collection plate, the angle of syringe with respect to the collection plate, and the applied voltage. Micro and nanofibers with wide ranges of diameters from 1-999 nm to within the micron range can be obtained by varying various experimental parameters such as viscosity of the polymer solution, electric potential at the capillary tip, diameter of the capillary tip as well as the gap or distance between the tip and the collecting screen.

For purposes of the present invention, “fiber” is meant to include fibrils ranging from nano- to micro-scales in diameter.

These fiber assemblies can be spun from any polymer which can be dissolved in a solvent. The solvent can be either organic or aqueous depending upon the selected polymer. Examples of polymers which can be used in production of the polymeric fibers of the present invention include, but are not limited to, biodegradable and/or bioabsorbable polymers such as poly(lactic acid-glycolic acid), poly(lactic acid), poly(glycolic acid), poly(glaxanone), poly(orthoesters), poly(pyrolic acid) and poly(phosphazenes), preferably containing a monomer selected from the group consisting of a glycolide, lactide, dioxanone, caprolactone, trimethylene carbonate, ethylene glycol and lysine. The biodegradable and/or bioabsorbable fiberizable material can also include a material derived from biological tissue, e.g., collagen, gelatin, polypeptides, proteins, hyaluronic acid and derivatives or synthetic biopolymers.

Micro and nanofibers of this invention may be formed of non-biodegradable or biodegradable polyphosphazenes, blends of polyphosphazenes with a biodegradable organic polymer (such as PLGA), or composite nanofibers from polyphosphazene and nanocrystals of hydroxyapatite (HAP) or nanoparticles such as Au or Ag, or polymeric nanoparticles with drug.

A bioactive compound may be incorporated within the polymeric fibers either by suspension of compound particles or dissolution of the compound in the solvent used to dissolve the polymer prior to electrospinning of the polymeric fibers. The bioactive compound may reside inside the fiber or may be dispersed between the fibers. Examples of bioactive compounds which can be incorporated into the polymeric fibers include, but are not limited to such pharmaceutical agents as steroids, antifungal agents, and anticancer agents. Other bioactive compounds of particular use in the present invention include tissue growth factors, angiogenesis factors, and anti-clotting factors.

If the bioactive compound is to reside within or inside the polymer fiber, selection of the polymer should be based upon the solubility of the bioactive compound within the polymer solution. Water soluble polymers such as polyethylene oxide can be used if the bioactive compound also dissolves in water. Alternatively, hydrophobic bioactive compounds which are soluble in organic solvent such as steroids can be dissolved in an organic solvent together with a hydrophobic polymer such as polylactic glycolic acid (PLGA).

If the bioactive compound is to reside between the polymer fibers, dissolution of the bioactive compound in the polymer solution is not required. Instead, the bioactive compound can be suspended in the polymer solution prior to electrospinning of the fibers.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

As used herein and in the appended claims, the singular forms “a”, “an”, and “the” include plural references unless the context clearly dictates otherwise. All technical and scientific terms used herein have the same meaning. Efforts have been made to ensure accuracy with respect to numbers used (e.g. amounts or temperature) but some experimental errors and deviations should be accounted for. Unless indicated otherwise, parts are parts by weight, molecular weight is weight average molecular weight, temperature is in degrees Centigrade, and pressure is at or near atmospheric.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The following examples are provided as follows: 1) preparation and evaluation of biomimetic fibers for the assembly of cells, 2) layer-by-layer assembly of cells into three-dimensional tissue structure. These examples show the feasibility of the present invention, using, but not limited to, polycaprolactone (PCL), collagen type I, bovine serum albumin (BSA) as model molecules. As stated above, these studies, including these model reagents and any other aspect of the examples, are not to be construed as limiting the invention in any way.

### Example 1

### Biomimetic Fibers for Layer-by-Layer Cell Building

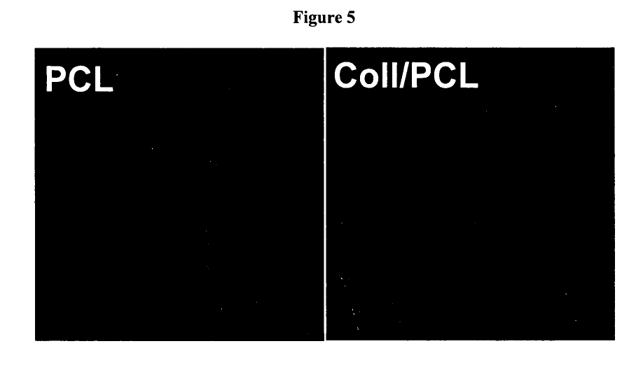

For preparation of suitable fibrous scaffolds supporting the growth and differentiation of bone cells and endothelial cells, we used an electrospinning technique, which produces fibers with similar dimensions as matrix fibrils and variable chemical compositions similar to those found in the ECM. In our preliminary study, collagen type I from calf skin was first electrospun into fibers with high mechanical properties by blending with polycaprolactone (PCL). The diameter of obtained fiber ranged from 50 nanometers to several micrometers, depending on polymer concentration and spinning conditions. Improved cell adhesion and proliferation of fibroblasts by collagen was observed, consistent with other studies (Liu, G., et al. Chin J Traumatol, 2004. 7(6): p. 358-62; Xiao, Y., et al. Tissue Eng, 2003. 9(6): p. 1167-77; Petrovic, L., et al. Int J Oral Maxillofac Implants, 2006. 21(2): p. 225-31). Fibroblasts cultured on collagen-based fibrous membrane showed better cell spreading morphology (FIG. 5), compared to PCL alone. Therefore, PCL/Collagen I (range at 1:1 to 4:1) was used in this study. Our studies show that these collagen I containing fibers support a variety of cell types such as osteoblasts, smooth muscle cells, keratinocytes, fibroblasts, endothelial cells, epithelial cells, among others.

### Example 2

### Layer-by-Layer Alternating Assembly of Multilayer-Cell Structure with Electrospun Fibers Sandwiched in Between

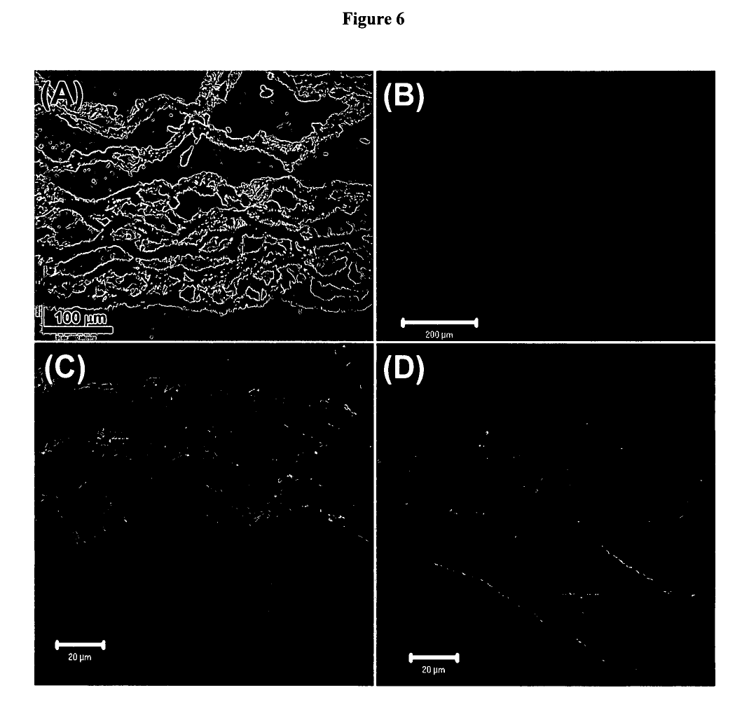

To test the feasibility of forming a multilayered structure, a study was done using human dermal fibroblast. This multilayer cell sheet with alternating layers of human fibroblasts (8 layers) and layers of collagen/PCL nanofibers (9 layers) was layer-by-layer fabricated as shown in FIG. 3. The space between cell layers was adjustable and defined by the thickness of nanofibrous layer. During the alternating cell layering, fibroblast culture medium (Dulbecco's modified minimum essential medium (DMEM) (Invitrogen) supplemented with 10% foetal bovine serum (FBS), 50 U/mL penicillin, 50 μg/mL streptomycin) is used. In the prepared cell sheet, the space between cell layers was around 5-25 μm. Closer examination of the cross section of cell sheet cultured for 2 days at 37° C. in CO₂ incubator stained with methylene blue under an optical microscope clearly showed the presence of multiple cell layers between electrospun fibers (FIG. 6). In addition, cells in the multi-layer constructs showed elongated morphology and embedded among fibers, similar as that in vivo, indicating the potential advantage of this new approach. Although the cells used were human dermal fibroblasts, the successful creation of multiple lamellar cell layers is conceptually a critical step towards layering any type of cells.

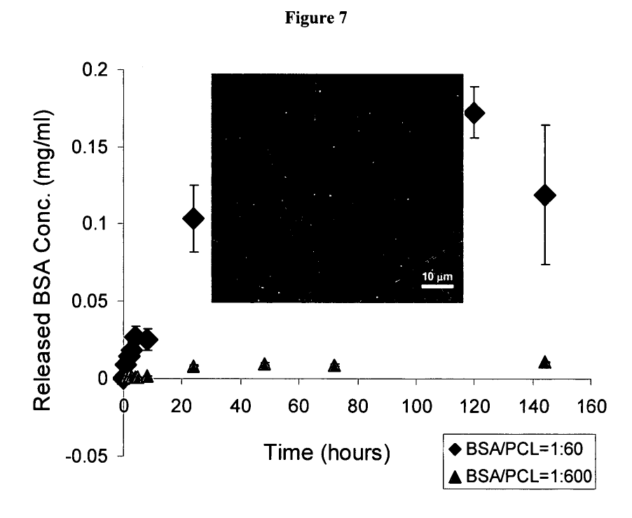

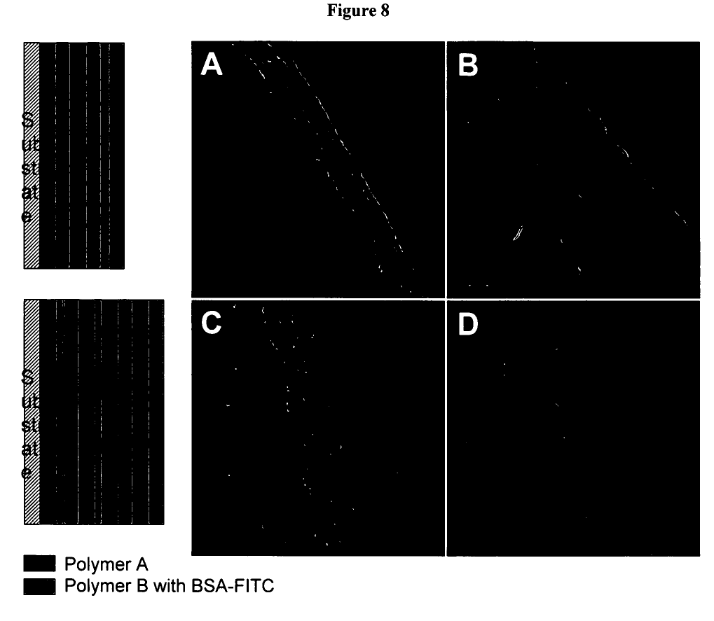

The proposed layer-by-layer cell assembly is a bottom up approach. During this assembly, non-woven fiber layers are used to accomplish this process. Electrospun fibers are advantageous for many reasons, e.g., geometrical similarities to ECM fibrils. Growth factors and cytokines are important in controlling cell activities (proliferation, migration, and differentiation). A long release duration and local delivery to targeted cells are necessary to reinforce the effect. To test the feasibility of incorporating bioactive molecules in the electrospun fibers, bovine serum albumin (BSA) was used as a model protein. Different amount of BSA was included in the polymer solution of PCL from 1:1 to 1:600 (BSA/PCL). Then BSA-containing PCL fibrous meshes were cut into discs (1.3 cm in diameter) and incubated in PBS at 37° C. under gentle shaking (40-80 rpm). The supernatant of incubation was collected at different times for protein measurement using the Lowry assay. For low BSA/PCL amount (1:300 and 1:600), fluorescence-labeled (FITC) BSA was used instead. The released BSA was measured indirectly by measuring the fluorescence intensity. Release results were shown in FIG. 7. Meanwhile, the distribution of BSA in the electrospun PCL fibers was homogeneous as shown in the inset of FIG. 7 using FITC-labeled BSA. The preliminary study showing a continuous release of BSA from electrospun fibers for more than 3 weeks is promising. To test the capability of manipulating the sequential deposition of different layers of fibers, a study was performed using PCL (Polymer A) only and PCL/Collagen (Polymer B) containing FITC-BSA. The sequence was shown as in the FIG. 8.

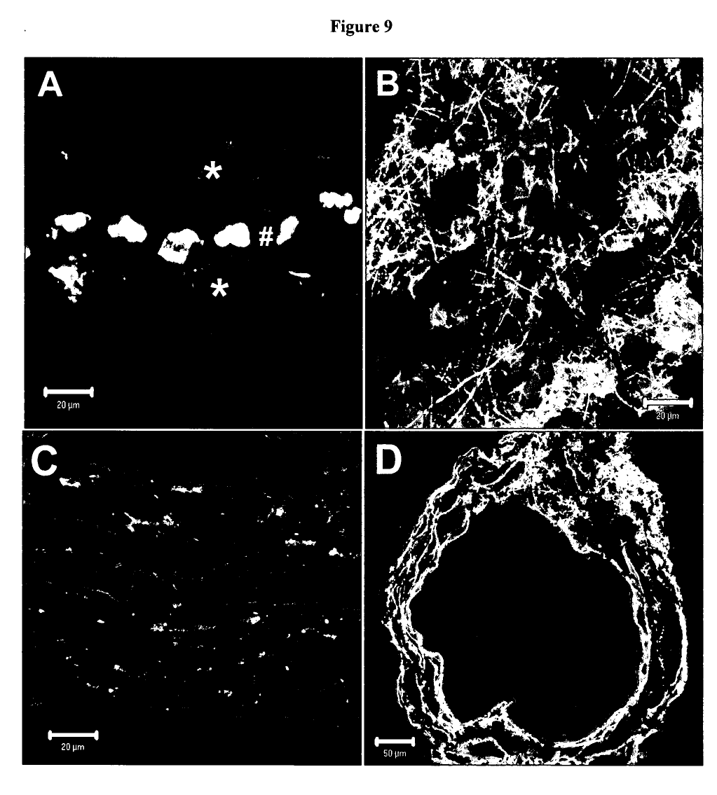

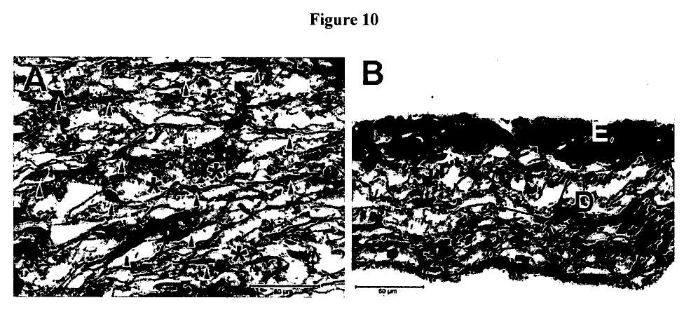

The possibility of selectively attaching cells to specific fiber layers using different materials was also investigated. For example, in FIGS. 9A and 9B, human dermal fibroblasts prefer to attach on the PCL/Collagen fibers instead of PCL only. The formed multilayer cell/fiber construct can be stacked to form thicker tissue (FIG. 9C) or rolled into tubular shape (FIG. 9D). By continue to culture the formed layered cell/fiber construct, dermal tissues (FIG. 10A) or skin tissues (FIG. 10B) can be obtained in short time.

The above examples demonstrate the present invention's innovative approach to tissue formation through a bottom-up layer-by-layer cell assembly, with marked potential to form functional tissues composed of multiple cell types, complex composition and vascularization. All examples (1-2) demonstrate that it is feasible to form a three-layer structure with cells sandwiched in between microparticle or micro/nanofiber layers.

The layer-by-layer cell assembly of this invention not only allows the co-culture of multiple cell types, but also provides a specifically formulated microenvironment with favorable composition and growth factors for certain cell types. Co-culturing multiple cell types for tissue engineering is preferable to culturing single cell types, and localized delivery of bioactive molecules such as growth factors to the specific cells can eliminate unwanted influence on other cell types. Thus, the layer-by-layer cell assembly approach of this invention, the feasibility of which has been demonstrated by the above examples, has many diverse future applications, especially those in scaffold design for tissue engineering.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the invention.

What is claimed is:

1. A synthetic tissue scaffold, the scaffold comprising alternating layers of electrospun polymers, optionally incorporating at least one bioactive molecule, and cells.

2. The polymers of claim 1, wherein the polymer is biocompatible.

3. The synthetic scaffold of claim 1, wherein the alternating layers of fibers is suitable for tissue engineering or for delivery of at least one therapeutic agent.

4. The method of claim 1, wherein the shape, size and thickness of scaffold is variable.

5. A method for generating a three-dimensional tissue, the method comprising the steps:
a.) electrospinning a layer of polymers according to claim 1,
b.) depositing a layer of the electrospun polymers onto an aqueous surface in an arbitrary shape,
c.) depositing a layer of one or more living mammalian cells onto the layer of electrospun polymers,
d.) repeating steps b and c until achieving desired three-dimensional tissue size,
wherein in step (c) the first type is optionally replaced with a different type of cell, thereby forming the three-dimensional tissue.

6. The method of claim 5, the method further comprising the step of, after repeating step b, depositing a layer of a different type of one or more living mammalian cells or nanosize or microsize particles, the layer of the first type of one or more living mammalian cells or particles and the layer of the different type of one or more living mammalian cells and particles thereby forming a heterogeneous three-dimensional tissue or structure.

7. The method of claim 21, wherein the living mammalian cells in step (b) are autologous to a subject or from different individual or species and the tissue is implanted into a subject in need thereof.

8. The method of claim 5, wherein the shape, size and thickness of tissue are variable and can be further processed to form other structure.
